(19) United States
(12) Patent Application Publication
(10) Pub. No.: US 2004/0152772 A1
(43) Pub. Date: Aug. 5, 2004

(54) 1-butyric acid derivatives and the use thereof

(76) Inventors: Erich Eigenbrodt, Linden (DE); Stefan Muellner, Langenfeld/Rheinland (DE); Sybille Mazurek, Linden (DE); Hugo Fasold, Frankfurt (DE)

Correspondence Address: HARNESS, DICKEY & PIERCE, P.L.C., P.O. BOX 8910, RESTON, VA, 20195, US

(21) Appl. No.: 10/471,854

(22) PCT Filed: Mar. 13, 2002
(86) PCT No.: PCT/EP02/02774

(30) Foreign Application Priority Data
Mar. 13, 2001 (DE) 101 12 925.4
Mar. 13, 2001 (DE) 101 12 924.6

(51) Int. Cl. A61K 31/275; A61K 31/215; A61K 31/195; A61K 31/19

(52) U.S. Cl. 514/519; 514/521; 514/529; 514/557; 514/569; 514/570; 514/567; 514/563

(57) ABSTRACT

Provided is a group of active substances capable of inhibiting the proliferation of cancer cells and inhibiting inflammation, as well as suppressing bodily defense reactions to control autoimmune diseases, transplant rejections, and acute and chronic inflammatory reactions. The disclosed active substances may be generally represented by Formula I, below,

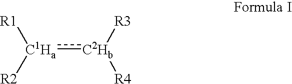

with R1-R4 comprising a range of substituents, and may be combined in a pharmaceutical composition with other active compounds and/or excipients, or delivered as a tautomer or physiologically tolerated salt of such a compound.

The invention pertains to derivatives of butanoic acid, pharmaceutical compositions containing such derivatives, and the uses of such derivatives for the manufacture of pharmaceutical compositions for the treatment of various diseases.

Cancer is one of the most frequent causes of death nowadays, and the number of cancer cases in the industrialized countries is increasing steadily. This is attributable, in particular, to the fact that malignant tumors are a disease of those of higher age, and more persons now reach this age thanks to the successful combating of infectious diseases. Despite all the advances in the diagnostic and therapeutic area, the prospects for healing are seldom more than 20% for the most frequently occurring internal forms of cancer. A cancer tumor can currently be destroyed, or inhibited in terms of its growth. The regression of a tumor cell into a normal cell cannot yet be achieved. The most important therapeutic procedures, namely operating and irradiating, remove cancer cells from the organism. Currently conventional chemotherapeutic cancer drugs, i.e. the cytostatic drugs, also lead merely to the destruction of, or damage to, tumor cells. In most cases, their action is so low in specificity that severe damage to healthy cells arises at the same time. In general, tumor cells exhibit a metabolism that is different from that of healthy cells, especially glycolysis. Thus a change in the isoenzyme system that is involved in glycolysis, and a change in the transportation of NADH are typical for tumor cells. Amongst other things, the activity of the glycolysis enzymes is increased. This also permits high extents of transformation under the aerobic conditions that are typical for tumor cells. Reference is made in detail in this regard to E. Eigenbrodt et al., Biochemical and Molecular Aspects of Selected Cancers, Vol. 2, pages 311 et seq., 1994.

### Prior Art

It is known from the literature reference E. Eigenbrodt et al., Biochemical and Molecular Aspects of Selected Cancers, Vol. 2, pages 311 et seq., 1994, that use can be made of glucose analogs in order to inhibit glycolysis. Other approaches that are known from here are the use of inhibitors of glycolytic enzymes, e.g. via suitable complex formation, or the inhibition of the formation of complexes. Tumor cells are, as it were, starved to death as a consequence. A problematic aspect in the case of the above compounds is that many of them are genotoxic, and/or insufficiently specific for tumor cells.

It is also known from the literature reference U. Mangold et al., Eur. J. Biochem., 266: 1-9, 1999, that, in combination with a new active substance to combat inflammatory diseases and to combat autoimmune reactions, these active substances, namely derivatives of leflunomide, intervene in glycolysis as well.

### Technical Problem for the Invention

The technical problem that forms the basis of the present invention is to provide active substances that are capable of inhibiting the proliferation of, in particular, cancer cells, and thus of inhibiting the growth of neoplastic tumors and of inhibiting inflammation, as well as inhibiting excessive defense reactions of the body such as e.g. septic shock, autoimmune diseases, transplant rejections, and acute and chronic inflammatory reactions, while simultaneously exhibiting only little or no cytotoxicity relative to the normal cells of the blood and of the immune system, and to the cells of tissues.

Thus the teaching of the invention relates to the use of a compound of Formula I in order to solve this technical problem

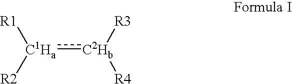

whereby a and b can be identical or different, and have values of 0 or 1,

whereby R1=—H, —C1-C18-alkyl, —C1-C18-cycloalkyl, or —C1-C18-aryl,

whereby R2=—OX1, —SX1, —COO⁻, —(CH₂)ₙ—COOX1 or —COOX1 with X1=—H, —C1-C18-alkyl, —C1-C18-cycloalkyl, or —C1-C18-aryl, and with n=1-8,

whereby R3=—CN, —COO⁻, —COOX2, —CO—X2, —CO—NHX2 with X2=—H, —C1-C18-alkyl, —C1-C18-cycloalkyl, or —C1-C18-aryl,

whereby R4=═O, —NHY, or —CONHZ with Y=—H, —CO—R (R=—C1-C18-alkyl, —C1-C18-cycloalkyl, or —C1-C18-aryl, or —NHA, with A=—H, —C1-C18-alkyl, —C1-C18-cycloalkyl, or —C1-C18-aryl), and Z=phenyl, naphthyl, or phenyl that is substituted by -Hal and/or —O-Hal and/or —C1-C8-alkyl, —C1-C8-cycloalkyl, or —C1-C8-aryl, or naphthyl that is substituted by —C1-C8-alkyl, —C1-C8-cycloalkyl, or —C1-C8-aryl (-Hal=—F, —Cl, or —Br),

whereby a and b correspond to the number of residual carbon valences at C¹ and C², whereby ring closure can take place to C¹ via R3 together with the elimination of X1 in R2 and X2 in R3,

or a physiologically tolerated salt of such a compound,

for the manufacture of a pharmaceutical composition for the treatment and/or prophylaxis of diseases from the group comprising neoplastic tumors, inflammatory diseases, autoimmune diseases, especially systemic lupus erythematosus, degenerative joint diseases, diseases of the rheumatic type with cartilage degradation, all the progressive forms of arthritis, especially rheumatoid and chronic polyarthritis, joint trauma, immobilization-engendered cartilage atrophy, septic shock, diseases with disrupted leucocyte adhesion, diseases as a result of increased TNF alpha concentrations, cachexia, Crohn's disease, skin psoriasis, Wegener granulatosis syndrome, rejection reactions following transplantations, especially within the context of cell therapy or stem cell therapy.

Some of the substances that are included within the above definition are known as such and from other connections. However, other substances that are included within the above definition are new. Thus the teaching of the invention also relates to compounds in accordance with Formula I

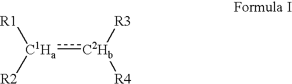

whereby a and b can be identical or different, and have values of 0 or 1,

whereby R1=—H, —C1-C18-alkyl, —C1-C18-cycloalkyl, or —C1-C18-aryl,

whereby R2=—OX1, —SX1, —COO⁻, —(CH₂)ₙ—COOX1 or —COOX1 with X1=—H, —C1-C18-alkyl, —C1-C18-cycloalkyl, or —C1-C18-aryl, and with n=1-8,

whereby R3=—CN,

whereby R4=═O, —NHY, or —CONHZ with Y=—H, —CO—R (R=—C1-C18-alkyl, —C1-C18-cycloalkyl, or —C1-C18-aryl, or —NHA, with A=—H, —C1-C18-alkyl, —C1-C18-cycloalkyl, or —C1-C18-aryl), and Z=phenyl, naphthyl, or phenyl that is substituted by -Hal and/or —O-Hal and/or —C1-C8-alkyl, —C1-C8-cycloalkyl, or —C1-C8-aryl, or naphthyl that is substituted by -Hal and/or —O-Hal and/or —C1-C8-alkyl, —C1-C8cycloalkyl, or —C1-C8-aryl (-Hal=—F, —Cl, or —Br),

whereby a and b correspond to the number of residual carbon valences at C¹ and C²,

or a physiologically tolerated salt of such a compound.

Finally, the teaching of the invention relates to a pharmaceutical composition containing a compound of Formula I

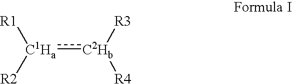

whereby a and b can be identical or different, and have values of 0 or 1,

whereby R1=—H, —C1-C18-alkyl, —C1-C18-cycloalkyl, or —C1-C18-aryl,

whereby R2=—OX1, —SX1, —COO⁻, —(CH₂)ₙ—COOX1 or —COOX1 with X1=—H, —C1-C18-alkyl, —C1-C18-cycloalkyl, or —C1-C18-aryl, and with n=1-8,

whereby R3=—CN, —COO⁻, —COOX2, —CO—X2, —CO—NHX2 with X2=—H, —C1-C18-alkyl, —C1-C18-cycloalkyl, or —C1-C18-aryl,

whereby R4=═O, —NHY, or —CONHZ with Y=—H, —CO—R (R=—C1-C18-alkyl, —C1-C18-cycloalkyl, or —C1-C18-aryl, or —NHA, with A=—H, —C1-C18-alkyl, —C1-C18-cycloalkyl, or —C1-C18-aryl), and Z=phenyl, naphthyl, or phenyl that is substituted by -Hal and/or —O-Hal and/or —C1-C8-alkyl, —C1-C8-cycloalkyl, or —C1-C8-aryl, or naphthyl that is substituted by -Hal and/or —O-Hal and/or —C1-C8-alkyl, —C1-C8cycloalkyl, or —C1-C8-aryl (-Hal=—F, —Cl, or —Br),

whereby a and b correspond to the number of residual carbon valences at C¹ and C², whereby ring closure can take place to C¹ via R3 together with the elimination of X1 in R2 and X2 in R3,

or a physiologically tolerated salt of such a compound,

and at least one physiologically tolerated ancillary substance, and/or vehicle substance.

It will be understood that different stereoisomers (especially enantiomers, and diastereomers) can possibly exist for the compounds in accordance with Formula I, whereby the subject of the invention comprises all of these. The term alkyl comprises linear and branched alkyl groups. The term cycloalkyl also comprises cycloalkyl groups with linear or branched alkyl substituents. The term aryl also comprises aralkyl groups, whereby the alkyl substituents can be alkyl or cycloalkyl.

Surprisingly it has been found that such derivatives of 1-butanoic acid of general Formula I are capable of inhibiting the proliferation in vitro of cancer cells in therapeutically relevant concentrations in a dose-dependent manner. No cytotoxic action could thereby be established in the dose range that was examined. Thus the compounds of general Formula I have an anti-proliferative action, i.e. the multiplication of tissues is retarded. In cases of chronic polyarthritis, for example, proliferation of the inner skin of joints arises, whereby this multiplies almost as fast as a tumor, and grows into the joint and destroys the cartilage and the bone with increasing duration of the disease. The anti-proliferative action of the compounds of Formula I in accordance with the invention brings about the direct retardation of such proliferation along with a reduction in so-called systemic inflammation activity, and wound healing or regeneration in all processes of proliferation within the context of inflammation. As a result of their pharmacological properties, the compounds in accordance with the invention are also, therefore, superbly well suited to the treatment and prophylaxis of the additional diseases that are enumerated above. The medical definitions and terms above can be gathered from the Roche Lexikon Medizin [Roche's Lexicon of Medicine], 4th edition, Munich, 1999.

In addition, the invention pertains to a diagnostic system containing at least one compound of Formula I for detecting diseases as designated above by bringing the cell or cell culture, which is to be examined, into contact with such a compound, and evaluating it in a suitable manner. See Example 2 in this regard.

### FORMS OF EMBODIMENT OF THE INVENTION

Various non-limitative forms of embodiment are possible within the framework of the invention. Thus a pharmaceutical composition in accordance with the invention can contain several different compounds that are included within the aforementioned definitions. Moreover, a pharmaceutical composition in accordance with the invention can additionally contain at least one active substance that differs from the compound of Formula I. One is then dealing with a combination preparation. In this case, the different active substances that are used can be prepared in one single form of agent for administration, i.e. the active substances are intermixed in this form of agent for administration. However, it is also possible to prepare the different active substances in spatially separated forms of agents for administration of the same or a differing type. Preferred active substances in this connection are the so-called immunomodulators, such as leflunomide (Arava®), methotrexate, or antirheumatic drugs.

It is preferred if the compounds in accordance with the invention have the following groups:

R1=—H, methyl, or ethyl,

R2=—OX, —COO⁻, or —COOX, with X1=—H, methyl, or ethyl,

R3=—CN, —COOH, —COO⁻, —COX2, —CO—NHX2, or whereby ring closure can take place to C¹ via R3 together with the elimination of X1 in R2 and X2 in R3,

R4=═O, —NHY, with Y=H or —COR (R=methyl, ethyl, or —NHA, with A=H, methyl, or ethyl) or CO—NHZ, with Z=—F, —Br, —Cl, or —O—Cl, and/or phenyl that has been substituted by —O—Br.

Especially suitable examples of compounds that are included under Formula I will be explained as follows.

Compound 1:

R1=methyl, R2=—OH, R3=—CN, R4=—NH₂, a=b=0

Compound 2:

R1=methyl, R2=—OH, R3=COOH, R4=—NH₂, a=b=0

Compound 3:

R1=methyl, R2=—OH, R3=—CN, R4=—NHY, a=b=0

Compounds 4-6:

R1=methyl, R2=—OH, R3=—CN, R4=—CO—NH—C₆H4F (e.g. meta), —CO—NH—C₆H₃Br₂ (e.g. ortho, meta), or C₆H₄OCl (e.g. para), a=b=0

Compound 7:

R1=methyl, R2=—OH, R3=—CN, R4=—CO—NH—Z, a=b=0

Compound 8:

R1=methyl, R2=—OH, R3=—CN, R4=—NH₂, a=b=0

Compound 9:

R1=—H, R2=—COO-methyl, R3=—CN, R4=═O, a=1, b=0

Compound 10:

R1=—H, R2=—COO, R3=—COOH, R4=═O, a=1, b=0

Compound 11:

R1=—H, R2=—COO, R3=—COOH, R4=—NH—CO—NH₂, a=b=1

Compound 12:

R1=—H, R2=—COO, R3=—COOH, R4=NH₂, a=b=1

Compound 13:

R1=—H, R2=—CH₂COO-methyl, R3=—CN, R4=═O, a=1, b=0

Compound 14:

R1=—H, R2=—OX1, R3=—CO—X2, R4=NH₂, a=b=1, X1 and X2 eliminated

Compound 15:

R1=—H, R2=—COOH, R3=—COOH, R4=—NH—CO—NH₂, a=b=1

Compound 16:

R1=—H, R2=—OX1, R3=—CO—NHX2, R4=NH₂, a=b=1, X1 and X2 eliminated.

Compounds of Formula I are quite especially preferred if they can exhibit (oxo-enol) tautomerism, such as the methyl ester of 4-cyano, 4-oxo-butanoic acid (Compound 9; formerly carbomethoxypropionyl cyanide).

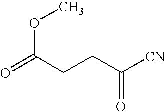

Consideration can be given to the following as counter ions for the ionic compounds of Formula I: Na⁺, K⁺, Li⁺, cyclohexylammonium or basic amino acids (e.g. lysine, arginine, omithine, glutamine). The medicinal drugs that are manufactured with compounds in accordance with the invention can be administered orally, intramuscularly, peri-articularly, intra-articularly, intravenously, intraperotoneally, subcutaneously, or rectally. The invention pertains to processes for the manufacture of medicinal drugs that are characterized by the feature that at least one compound of Formula I is brought into a suitable form of agent for administration together with a pharmaceutically suitable and physiologically tolerated vehicle and, optionally, further suitable active substances, additives, or ancillary substances. Suitable solid or liquid galenic forms of preparation or formulations are, for example, granulated materials, powders, sugar-coated pills, tablets, (micro)capsules, suppositories, syrups, juices, suspensions, emulsions, drops, or injectable solutions as well as preparations with a protracted release of the active substance, whereby use is made in their preparation of conventional ancillary substances, such as vehicle substances, agents that lead to the disintegration of the preparation, binders, coating agents, swelling agents, slippage promoting agents or lubricants, taste improving agents, sweeteners, and solubilizers. Mention may be made of the following as ancillary substances: magnesium carbonate, titanium dioxide, lactose, mannitol and other sugars, talcum, milk protein, gelatine, starch, cellulose and its derivatives, animal and vegetable oils such as cod-liver oil, sun flower oil, groundnut or sesame oil, poly(ethylene glycols), and solvents such as, for example, sterile water and monohydric or polyhydric alcohols, e.g. glycerine.

The medicinal drugs are preferably manufactured and administered in dosage units, whereby each unit contains, as the active component, a defined dose of the compound according to Formula I in accordance with the invention. In the case of solid dosage units, such as tablets, capsules, sugar-coated pills or suppositories, this dose can amount to 1 to 1000 mg and preferably 50 to 300 mg, and in the case of injection solutions in ampoule form, this dose can amount to 0.3 to 300 mg and preferably 10 to 100 mg.

Daily doses of 20 to 1000 mg of active substance, and preferably 100 to 500 mg of active substance, are indicated for the treatment of an adult patient weighing 50 to 100 kg, e.g. 70 kg. However, higher or lower daily doses can also be applied under certain circumstances.

The administration of the daily dose can take place via an administration on one single occasion in the form of an individual dosage unit or several smaller dosage units, or via the multiple administration of subdivided doses at defined intervals.

The invention will be explained in more detail in the following section by means of examples that merely represent forms of embodiments.

### EXAMPLE 1

The compound carbomethoxypropionyl cyanide was manufactured analogously to the method described by Q. Tang and S. Sen (Tetrahedron Letters 39, 1998, pp. 2249-2252). 1.5 g (10 mmol) of carboxymethylpropionyl chloride was typically added to a solution of 1.79 g of CuCN (20 mol) in 10 mL of acetonitrile. The mixture was heated under reflux for 30 minutes and, after cooling to room temperature, it was concentrated by evaporation on a Rotavapor. The residue was dissolved in ether, and the ether solution was filtered. A slightly yellow oil remained behind after removing the solvent (yield 0.95 g, 67% of theory); IR (cm⁻¹) 2225, 1727.

### EXAMPLE 2

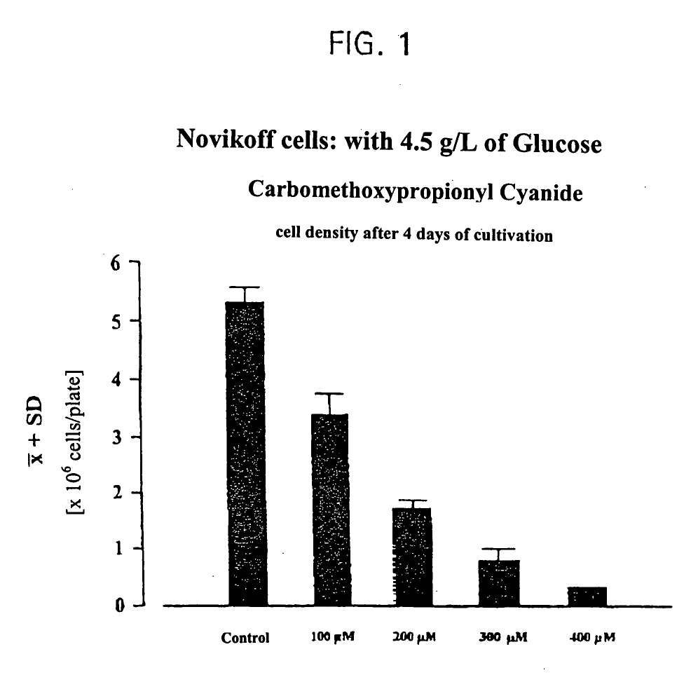

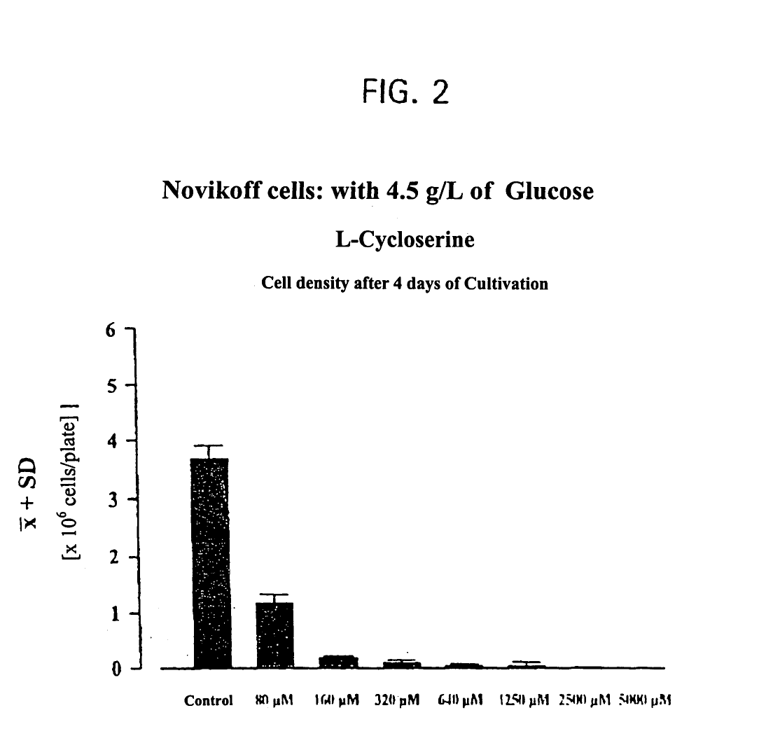

The Novikoff hepatoma cells that were used derived from the Tumor Bank of the German Cancer Research Center, Heidelberg (Cancer Research 1951, 17, 1010). 100,000 cells were spread out per 25 cm² of cultivation surface on each occasion. After having been dissolved in a solvent, such as e.g. water, diluted ethanol, dimethyl sulfoxide or a similar material that is suitable for use in cell cultures, the substance in accordance with the invention according to Example 1, or L-cycloserine, or dehydrothreonine was added to the culture medium in increasing concentrations, e.g. L-cycloserine (Compound 16) or dehydrothreonine (Compound 2) in the range of concentrations from 80 μM-5000 μM; and carbomethoxypropionyl cyanide (Compound 13) in the range of concentrations from 100 μM-300 μM. The cell count per flask was enumerated after four days of cultivation. The results are reproduced in FIGS. 1. and 2, and a dose-dependent inhibition of proliferation is seen in comparison to the control sample without an addition of a compound in accordance with the invention.

### EXAMPLE 3

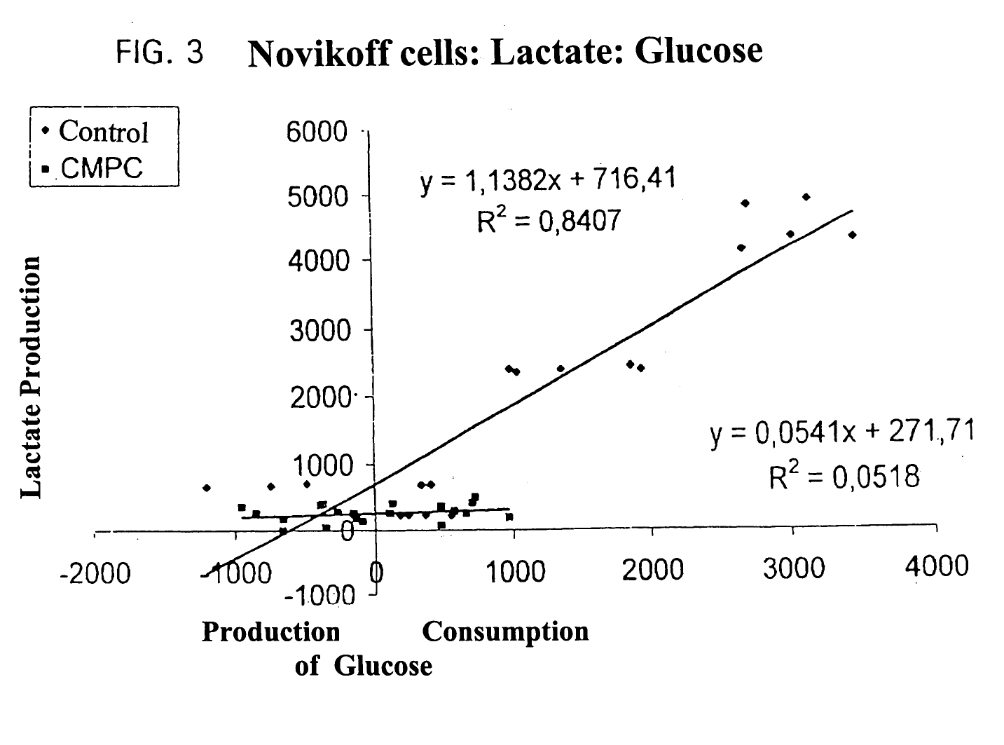

The investigations of carbomethoxypropionyl cyanide (CMPC) on the metabolism of Novikoff cells resulted in the finding that CMPC massively inhibited the flow of glycolysis as will be seen from a consideration of FIG. 3.

1. Use of a compound of Formula I 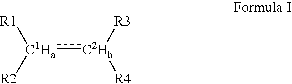
whereby a and b can be identical or different, and have values of 0 or 1,
whereby R1=—H, —C1-C18-alkyl, —C1-C18-cycloalkyl, or —C1-C18-aryl,
whereby R2=—OX1, —SX1, —COO⁻, —(CH₂)ₙ—COOX1 or —COOX1 with X1=—H, —C1-C18-alkyl, —C1-C18-cycloalkyl, or —C1-C18-aryl, and with n=1-8,
whereby R3=—CN, —COO⁻, —COOX2, —CO—X2, —CO—NHX2 with X2=—H, —C1-C18-alkyl, —C1-C18-cycloalkyl, or —C1-C18-aryl,
whereby R4=═O, —NHY, or —CONHZ with Y=—H, —CO—R (R=—C1-C18-alkyl, —C1-C18-cycloalkyl, or —C1-C18-aryl, or —NHA, with A=—H, —C1-C18-alkyl, —C1-C18-cycloalkyl, or —C1-C18-aryl), and Z=phenyl, naphthyl, or phenyl that is substituted by -Hal and/or —O-Hal and/or —C1-C8-alkyl, —C1-C8-cycloalkyl, or —C1-C8-aryl, or naphthyl that is substituted by -Hal and/or —O-Hal and/or —C1-C8-alkyl, —C1-C8cycloalkyl, or —C1-C8-aryl (-Hal=—F, —Cl, or —Br),
whereby a and b correspond to the number of residual carbon valences at C¹ and C², whereby ring closure can take place to C¹ via R3 together with the elimination of X1 in R2 and X2 in R3,
or a physiologically tolerated salt of such a compound, for the manufacture of a pharrnaceutical composition for the treatment and/or prophylaxis of diseases from the group comprising neoplastic tumors, inflammatory diseases, autoimmune diseases, especially systemic lupus erythematosus, degenerative joint diseases, diseases of the rheumatic type with cartilage degradation, all the progressive forms of arthritis, especially rheumatoid and chronic polyarthritis, joint trauma, immobilization-engendered cartilage atrophy, septic shock, diseases with disrupted leucocyte adhesion, diseases as a result of increased TNF alpha concentrations, cachexia, Crohn's disease, skin psoriasis, Wegener granulatosis syndrome, rejection reactions following transplantations, especially within the context of cell therapy or stem cell therapy.

2. Use of a compound in accordance with Formula I 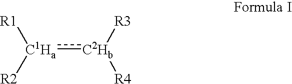
whereby a and b can be identical or different, and have values of 0 or 1,
whereby R1=—H, —C1-C18-alkyl, —C1-C18-cycloalkyl, or —C1-C18-aryl,
whereby R2=—OX1, —SX1, —COO⁻, —(CH₂)ₙ—COOX1 or —COOX1 with X1=—H, —C1-C18-alkyl, —C1-C18-cycloalkyl, or —C1-C18-aryl, and with n=1-8,
whereby R3=—CN,
whereby R4=═O, —NHY, or —CONHZ with Y=—H, —CO—R (R=—C1-C18-alkyl, —C1-C18-cycloalkyl, or —C1-C18-aryl, or —NHA, with A=—H, —C1-C18-alkyl, —C1-C18-cycloalkyl, or —C1-C18-aryl), and Z=phenyl, naphthyl, or phenyl that is substituted by -Hal and/or —O-Hal and/or —C1-C8-alkyl, —C1-C8-cycloalkyl, or —C1-C8-aryl, or naphthyl that is substituted by -Hal and/or —O-Hal and/or —C1-C8-alkyl, —C1-C8cycloalkyl, or —C1-C8-aryl (-Hal=—F, —Cl, or —Br),
whereby a and b correspond to the number of residual carbon valences at C¹ and C², or a physiologically tolerated salt of such a compound.

3. Pharmaceutical composition containing a compound of Formula I 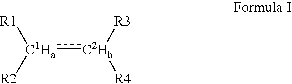
whereby a and b can be identical or different, and have values of 0 or 1,
whereby R1=—H, —C1-C18-alkyl, —C1-C18-cycloalkyl, or —C1-C18-aryl,
whereby R2=—OX1, —SX1, —COO⁻, —(CH₂)ₙ—COOX1 or —COOX1 with X1=—H, —C1-C18-alkyl, —C1-C18-cycloalkyl, or —C1-C18-aryl, and with n=1-8,
whereby R3=—CN, —COO⁻, —COOX2, —CO—X2, —CO—NHX2 with X2=—H, —C1-C18-alkyl, —C1-C18-cycloalkyl, or —C1-C18-aryl,
whereby R4=═O, —NHY, or —CONHZ with Y=—H, —CO—R (R=—C1-C18-alkyl, —C1-C18-cycloalkyl, or —C1-C18-aryl, or —NHA, with A=—H, —C1-C18-alkyl, —C1-C18-cycloalkyl, or —C1-C18-aryl), and Z=phenyl, naphthyl, or phenyl that is substituted by -Hal and/or —O-Hal and/or —C1-C8-alkyl, —C1-C8-cycloalkyl, or —C1-C8-aryl, or naphthyl that is substituted by -Hal and/or —O-Hal and/or —C1-C8-alkyl, —C1-C8cycloalkyl, or —C1-C8-aryl (-Hal=—F, —Cl, or —Br),
whereby a and b correspond to the number of residual carbon valences at C¹ and C², whereby ring closure can take place to C¹ via R3 together with the elimination of X1 in R2 and X2 in R3, or a physiologically tolerated salt of such a compound, and at least one physiologically tolerated ancillary substance, and/or vehicle substance.

4. Pharmaceutical composition in accordance with claim 3, whereby this composition contains at least one active substance, preferably leflunomide, methotrexate or antirheumatic drugs, differing from the compound of Formula I.

5. Use of a compound or pharmaceutical composition in accordance with one of the claims 1 through 4,
whereby
R1=—H, methyl, or ethyl,
R2=—OX 1, —COO—, or —COOX1, with X1=—H, methyl, or ethyl,
R3=—CN, —COOH, —COO⁻, —COX2, —CO—NHX2, whereby ring closure can take place to C¹ via R3 together with the elimination of X1 in R2 and X2 in R3,
R4=═O, —NHY, with Y=H or —CO—R (R=methyl, ethyl, or —NHA, with A=H, methyl, or ethyl) or CO—NHZ, with Z=—F, —Br, —Cl, or —O—Cl, and/or phenyl that has been substituted by —O—Br.

6. Compound according to Formula I in accordance with one of the claims 1 through 4, characterized by the feature that this compound possesses anti-proliferative action.

7. Compound according to Formula I in accordance with claim 6, characterized by the feature that this compound possesses inflammation inhibiting action.

8. Diagnostic system containing at least one compound according to Formula I in accordance with claim 1.
